(19) United States
(12) Patent Application Publication
(10) Pub. No.: US 2006/0100479 A1
(43) Pub. Date: May 11, 2006

(54) Implantable medical device for controlled release of substance

(76) Inventors: Daniel Yachia, Herzliya (IL); Eran Hirszowicz, Ramat Gan (IL)

Correspondence Address: NATH & ASSOCIATES, 112 South West Street, Alexandria, VA, 22314, US

(21) Appl. No.: 10/533,639

(22) PCT Filed: Nov. 4, 2003
(86) PCT No.: PCT/IL03/00914

§ 371 Date: Nov. 15, 2005

(30) Foreign Application Priority Data
Nov. 4, 2002 (IL) 152629

(51) Int. Cl. A61F 2/02

(52) U.S. Cl. 600/30

(57) ABSTRACT

A medical device for controlled release of one or more substances into a body cavity containing an electrolytic fluid, such as a urinary bladder. The device comprises a plurality of blister-like vesicles mounted on a first surface, each vesicle having at least a metallic portion formed from a first metal. The metallic portion of each vesicle is electrically connected to a first terminal of a power supply via a switch, so as to allow the metallic portion to function as an anode when the switch is closed. A cathode is formed from a second metal and is attached to the second terminal of the power supply The cathode is separated from the anodes by a space that is assessable by the electrolytic fluid when the device is in the body cavity.

### FIELD OF THE INVENTION

The invention is in the field of implantable medical devices. More specifically, the invention relates to such devices controlled release of a substance in a body cavity such as a urinary bladder or digestive tract organ.

### BACKGROUND OF THE INVENTION

There are many instances when it is desirable to release a substance in a body cavity over a prolonged period of time.

U.S. Pat. No. 6,364,856 to Ding et al., for example, discloses medical devices comprising an expandable portion which is covered with a sponge coating for releasing a biologically active material. The sponge coating is made of a non-hydrogel polymer having a plurality of voids. The device can further include means for infusing or expelling the biologically active material or drug into the voids. The drug is delivered to the body lumen of a patient by expelling the drug and inflating or expanding the expandable portion of the catheter or device.

U.S. Pat. No. 6,187,038 to Sullivan et al., discloses a composite graft for a blood vessel comprising: an inner vessel made of a biologic collagenic material and an outer sleeve surrounding the inner vessel and defining an annular gap between the inner vessel and the sleeve. A polymeric fabric, and a bioactive compound in the annular gap carried on a time-release vehicle.

U.S. Pat. No. 6,187,768 to Harle discloses corpuscles for implantation into or at body tissue. Medicine is distributed in carriers formed of biologically inert material for release into body tissue after implantation. The surface-to-volume ratio of the carriers is more satisfactory than that of the carriers forming part of conventional corpuscles so that they are readily withdrawn from the body.

U.S. Pat. Nos. 6,293,923 and 6,398,718 to Yachia et al describe devices for insertion into a urinary bladder that may be adapted to release a substance in the bladder.

While some prior art devices allow the rate at which a substance is released from the device to be determined, prior art devices do not allow the release of the substance to be started and stopped repeatedly over time as may be required in any particular application.

In its first aspect, the invention thus provides a medical device for controlled release of one or more substances into a body cavity containing an electrolytic fluid comprising:
(a) a power supply having first and second terminals;
(b) a plurality of blister-like vesicles mounted on a first surface, each vesicle having at least a metallic portion formed from a first metal;
(c) for each vesicle, an electrical connection between the metallic portion of the vesicle and the first terminal of the power supply, each connection including a switch so as to allow the metallic portion to function as an anode when the switch is closed; and
(d) A cathode formed from a second metal attached to the second terminal of the power supply;

wherein the cathode is separated from the anodes by a space that is assessable by the electrolytic fluid when the device is in the body cavity.

In its second aspect, the invention thus provides a system for treating a body cavity of an individual, the system comprising:
(a) a device according to the invention and
(b) an applicator for inserting the device into the body or for removing the device from the body cavity, the applicator fitted at an end thereof with a gripping device for releasably gripping the device;

In its third aspect, the invention thus provides a system for treating a body cavity of an individual, the system comprising:
(a) a device according to the invention;
(b) an applicator for inserting the device into the body or for removing the device from the body cavity, the applicator fitted at an end thereof with a gripping device for releasably gripping the device; and
(c) an inflating device for inflating the balloon.

In its fourth aspect, the invention thus provides a method for releasing one or more substances into a body cavity containing an electrolytic fluid of an individual comprising the steps of:
(a) loading the one or more substances into the vesicles of a device according to the invention;
(b) inserting the device into the body cavity;
(c) expanding the balloon in the urinary bladder; and
(d) displacing the balloon within the urinary bladder to a desired location.

In its fifth aspect, the invention thus provides a method for releasing one or more substances into a body cavity containing an electrolytic fluid of an individual comprising the steps of:
(a) loading the one or more substances into the vesicles of a device according to the invention;
(b) inserting the device into the body cavity; and
(c) expanding the balloon in the body cavity.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a device for releasing substances in a body cavity in accordance with one embodiment of the invention;

FIG. 2 shows a device for releasing substances in a body cavity in accordance with another embodiment of the invention;

FIG. 3 shows the embodiment of FIG. 2 mounted on an inflatable balloon;

FIG. 4 shows the balloon of FIG. 3 without the device;

FIG. 5 shows a portion of a balloon having a duck-bill valve;

FIG. 6 shows a portion of a balloon according to the invention having a ball valve;

FIG. 7 shows a device-balloon combination in which the balloon filled after have been inserted into body cavity;

FIG. 8 shows a device-balloon combination in which the balloon is filled before being inserted into the urinary bladder;

FIG. 9 shows use of an applicator for inserting a device-balloon combination into the urinary bladder of a female individual;

FIG. 10 shows use of an applicator for inserting a device-balloon combination into the urinary bladder of a male individual;

FIG. 11 shows a retrieval device for retrieving a device-balloon combination;

FIG. 12 shows use of a displacing member to position a device-balloon combination in a desired position in a body cavity; and

FIG. 14 shows use of an immobilizing member.

### DETAILED DESCRIPTION OF SPECIFIC EMBODIMENTS

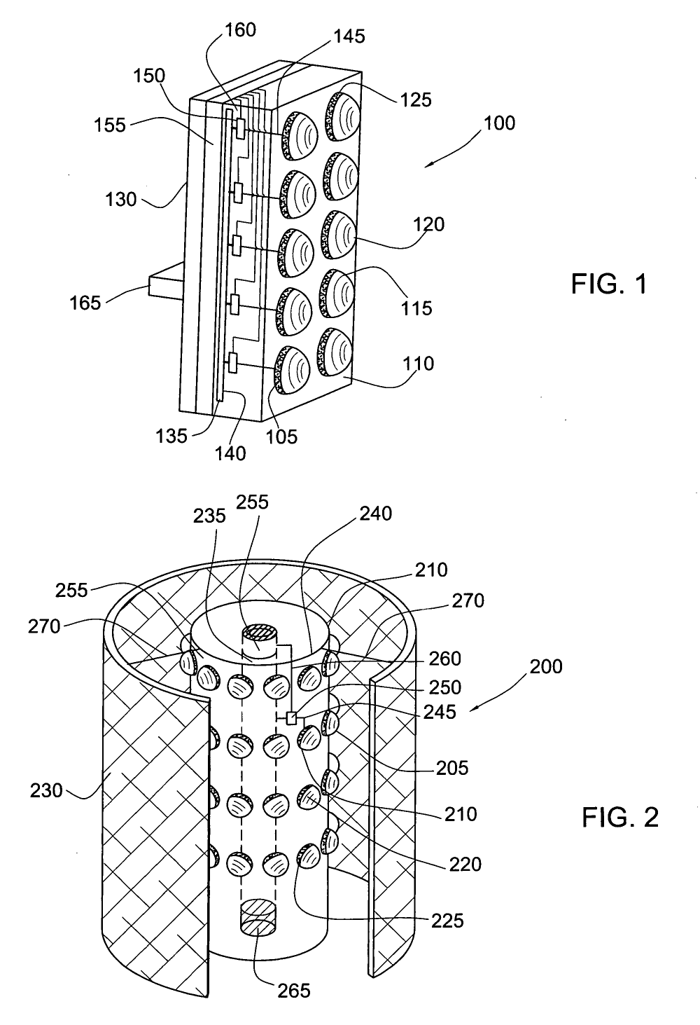

Reference is now made to FIG. 1 which shows a first embodiment 100 of a device for controlled released of a substance or substances into a body cavity in accordance with the invention. A plurality of blisters 105 are mounted on a first surface 110 of the device. The first surface 110 is formed from an insulating material such as rubber. Each blister 105 has a wall 115 surrounding a lumen 120. The lumen 120 of each blister is filled with the one or more substances (not shown) that are to be released into the body cavity. The wall 115 of each blister may be made entirely of metal, or may have a metallic portion. In the embodiment shown in FIG. 1, each blister has a metallic portion 125 that is referred to herein as the “anode”. A second surface 130 of the device (referred o herein as the “cathode”) 100 is formed from a second metal.

The device further comprises a power supply 135. Each anode 125 is connected to a first terminal 140 of the power supply 135 via an individual electrical connection 145. The connection 145 includes a switch 150. The cathode 130 is connected to the second terminal of the power supply 135.

The device may further include a processor 155 that is connected to each of the switches 145 by an individual connection 160 and is configured to close each switch at a predetermined time. For example, the processor 155 may be configured to close one switch every four hours, or some other predetermined time. Alternatively, the switches may be closed by means of a remote control located outside the body (not shown). For example, the user may be instructed to use the remote control to close a switch every evening before going to bed. AS yet another example, the device 100 may include one or more detectors (not shown) for monitoring conditions within the body cavity that are input to the processor, the processor closing a switch under predetermined conditions inside the body cavity.

The device 100 may also include a magnet 165 in order to allow the device 100 to be positioned in the body cavity by means of a second magnet located outside the body (not shown).

FIG. 2 shows another embodiment 200 of the invention. In this embodiment a plurality of blisters 205 are mounted on a cylindrical surface 210. The cylindrical surface 210 is formed from an insulating material such as rubber. Each blister 205 has a wall 215 surrounding a lumen 220. The lumen 220 of each blister is filled with the one or more substances (not shown) that are to be released into the body cavity. The wall 215 of each blister may be made entirely of metal, or may have a metallic portion. In the embodiment shown in FIG. 2, each blister has a metallic portion 225 that is referred to herein as the “anode”.

In the interior of the cylindrical surface 210 is a power supply 235. Each anode 225 is connected to a first terminal 240 of the power supply 235 via an individual electrical connection 245. The connection 245 includes a switch 250.

The interior of the cylindrical surface may further include a processor 255 that is connected to each of the switches 245 by an individual connection 260 and is configured to close each switch at a predetermined time. Alternatively, the switches may be closed by means of a remote control located outside the body (not shown).

Surrounding the cylindrical surface 240 is a cylindrically shaped cathode 230 that is coaxial with the cylindrical surface 240. This arrangement is maintained by means of rigid radial rods 270. The cathode 230 is formed from a second metal, and preferably has a mesh-like structure. The cathode 230 is connected to the second terminal of the power supply 135. The

The device 200 may also include a magnet 265 in order to allow the device 200 to be positioned in the body cavity by means of a second magnet located outside the body (not shown).

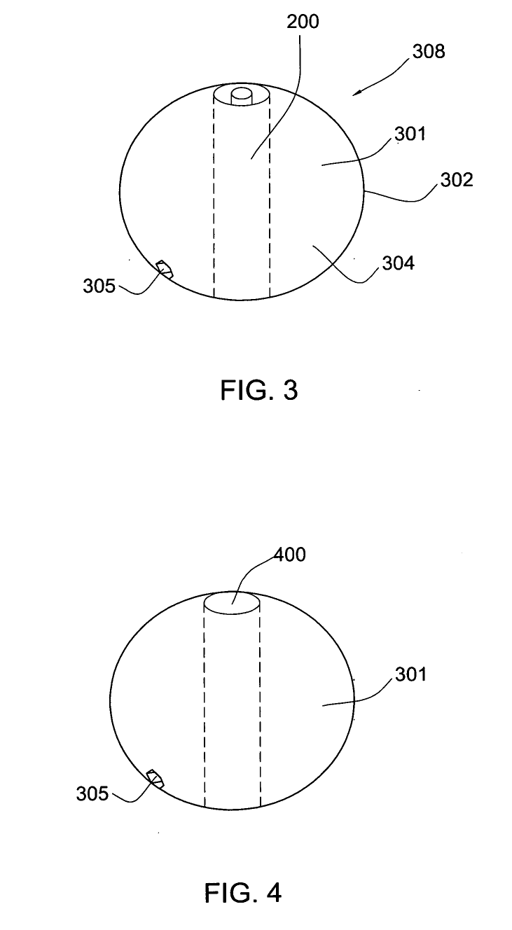

As shown in FIG. 3, the device 200 may be mounted on an inflatable balloon 301. The balloon 302 made of a bio-compatible material enclosing a lumen 204. The inflated balloon may have any desired shape as required in any particular application. In a preferred embodiment, the balloon 301 has a torroidal shape as shown in FIG. 3. The balloon 301 is shown in FIG. 4 without the device 200 being mounted in it. The balloon encircles a cylindrical hole 400 that is dimensioned to receive the device 200. The device 200 is firmly set in the hole 400 by means of pressure exerted on the device 200 by the wall 202 of the balloon 301 when inflated.

The lumen 304 of balloon 301 may be filled with a bio-compatible fluid which may be pre-sterilized such as air, water, saline or an oil such as liquid paraffin. The balloon 301 may further comprise a magnetizable portions (not shown) in order to position the device in the body cavity by means of n external magnet. The magnetizable portion may consist for example, of one or more metal particles which may be free in the lumen 304, attached to the inner surface of the wall 302 or embedded in the wall 302.

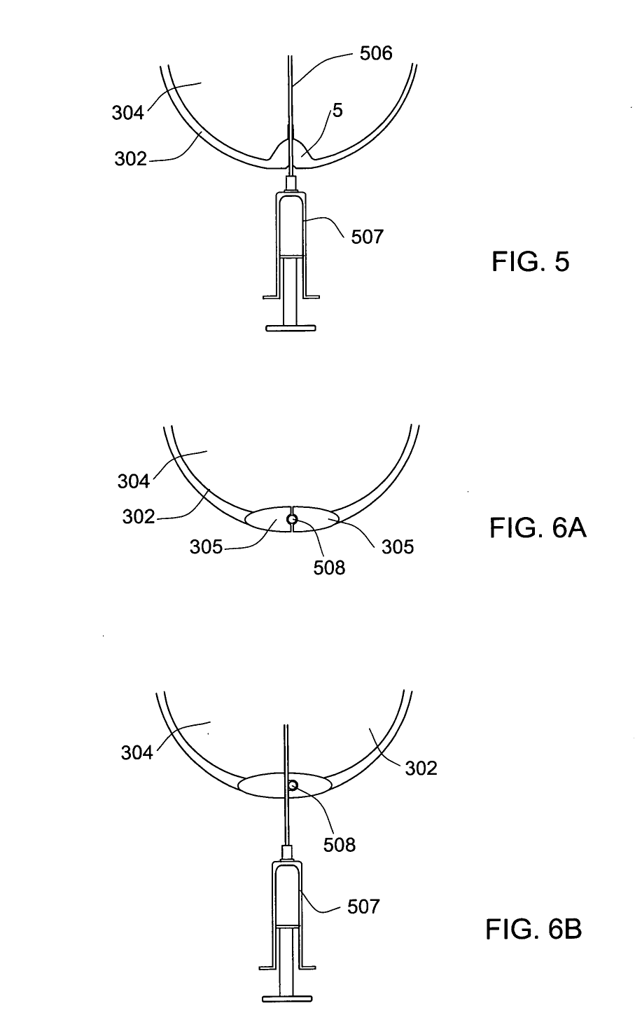

A self-sealing valve 305 in the wall of the balloon is used to fill the balloon. The valve 305 may be for example a duck-bill type valve as shown in FIG. 5, or a ball valve as shown in FIG. 6 in which a ball 508 may be in a sealing position (FIG. 6a) or an unsealing position (FIG. 6c). The canula 506 of a syringe 507 is inserted through the valve 305 into the lumen 304 of the balloon. Fluid injected into the lumen 304 causes the balloon 301 to expand. After filling, the syringe needle 506 is withdrawn, and the valve 305 seals itself. The inflated balloon with the device 200 mounted on it may float or sink in the electrolytic liquid in the body cavity.

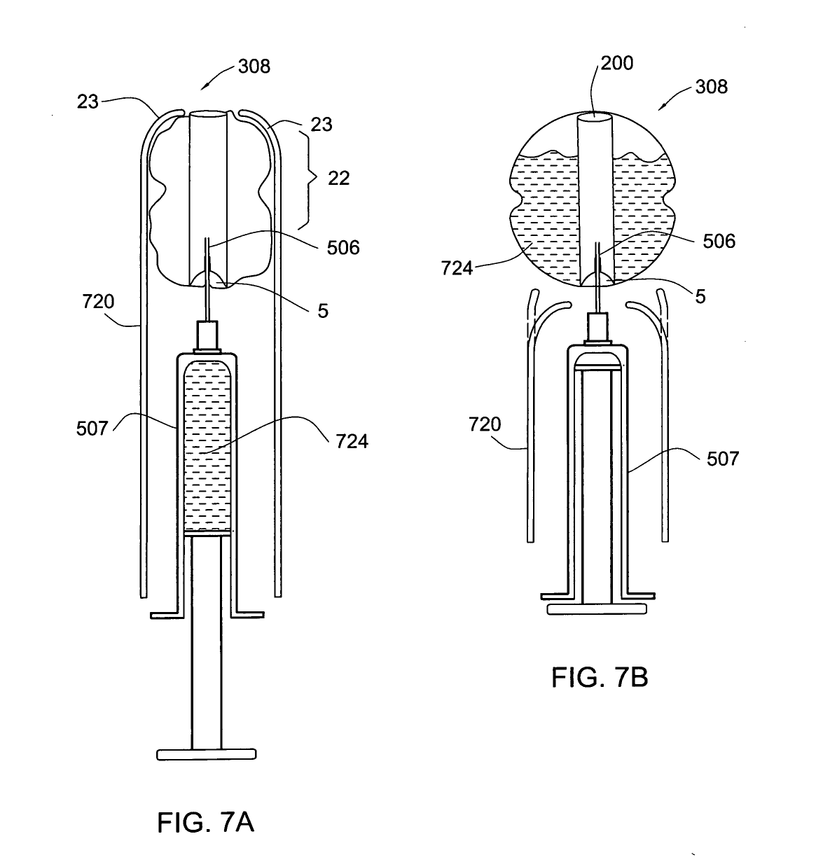

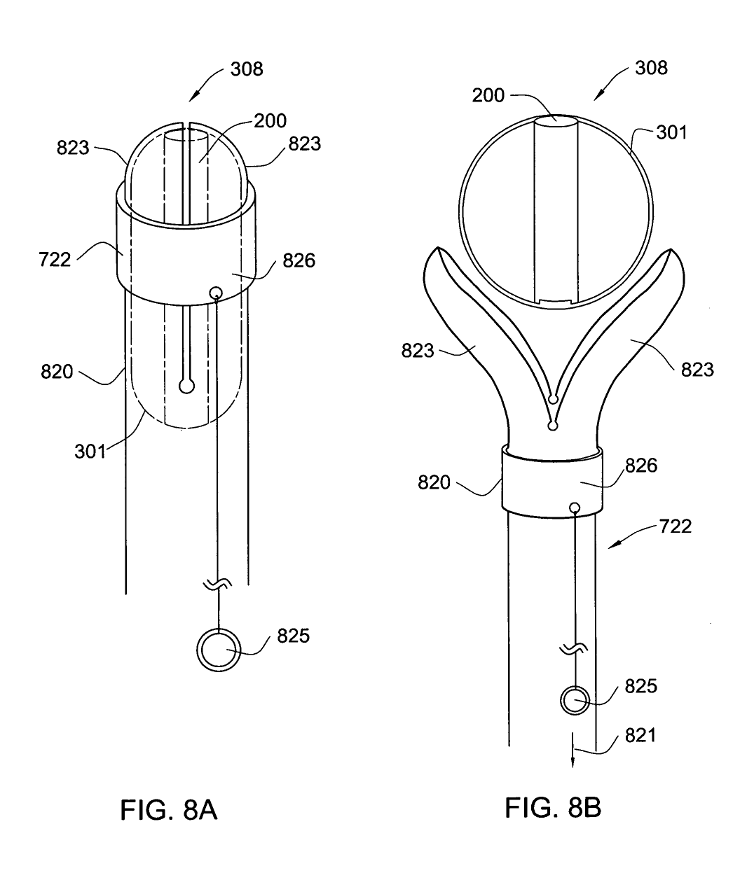

As shown in FIG. 7, the device-balloon combination 308 may first be delivered to the body cavity with the balloon 301 deflated, by means of an applicator 720 to be described below in detail (FIG. 7a). Following release of the device-balloon combination 308 from the applicator 720 into the cavity, the balloon 301 is filled with fluid 724 from the syringe 507 (FIG. 4b). Alternatively, as shown in FIG. 8a, the balloon 301 of the device-balloon combination 308 may be filled with a compressible fluid. The balloon 301 is then compressed before being inserted into the bladder by means of an applicator 820. The devie-balloon combination 308 with the pre-filled balloon is clutched by the flanges 823 which are initially kept closed by constraining sleeve 826 (FIG. 5a). After insertion of the applicator 820 with the device-balloon combination 308 into the body cavity, ring 825 is pulled as indicated by arrow 121 in FIG. 8b to urge the constraining sleeve 826 away from the flanges 823, allowing flanges 823 to open and release the device-balloon combination 308 with the pre-filled balloon 301 into the body cavity.

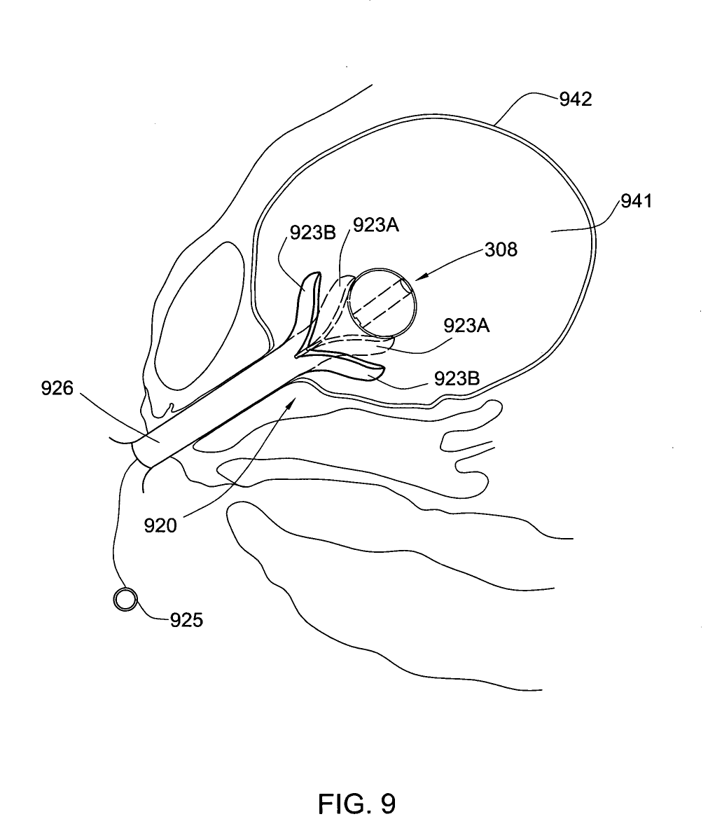

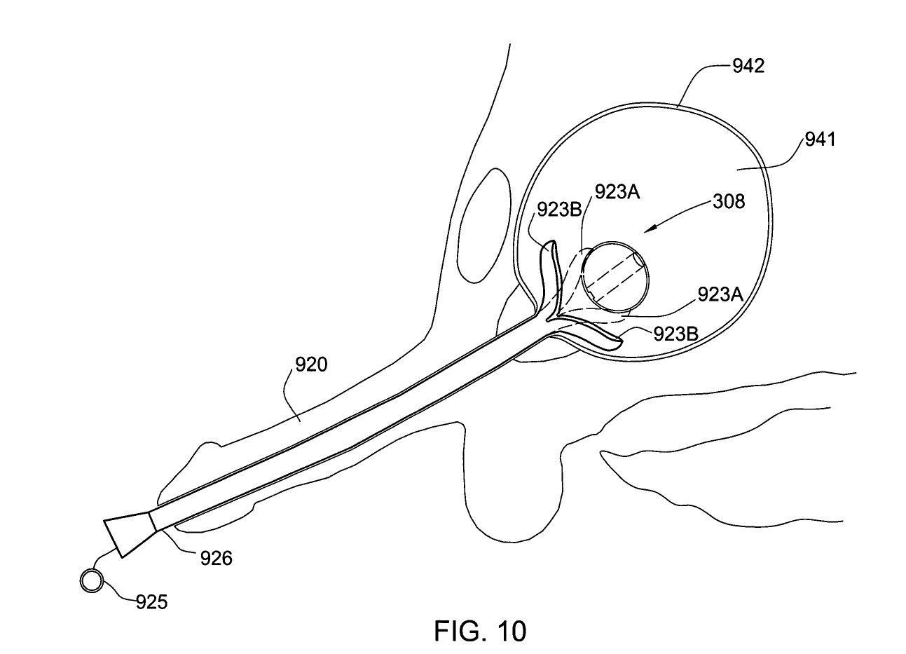

FIG. 9 shows use of an applicator 920 for inserting the device-balloon combination 308 into the lumen 941 of a urinary bladder 942 of a female individual, and FIG. 10 shows use of the applicator 920 inserting the device-balloon combination 308 into the lumen of the urinary bladder 942 of a male individual. In either case the device-balloon combination 308 is initially grasped by the closed flanges 923a at the distal end of the applicator 920 (FIGS. 9a and 10a). The distal end of the applicator with the device-balloon combination 308 is inserted into the urethra until it reaches the lumen 941 of the bladder 942. The device-balloon combination 308 is then released from the applicator by opening the flanges 923b by pulling on ring 925 while holding the constraining sleeve 926. The applicator 920 is then removed from the body, leaving the device-balloon combination in the bladder lumen 41.

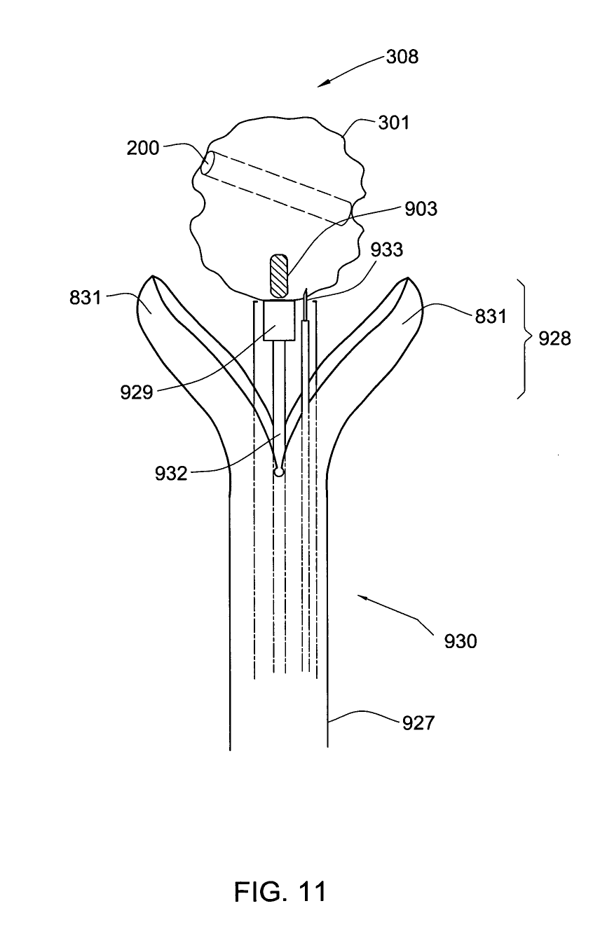

FIG. 11 shows a retrieval device generally designated as 930 for removing the device-balloon combination 308 from a body cavity. A catheter 927 has at its distal end 928 a magnetizable portion 929 so as to hold the device-balloon combination 308 at the distal tip 928 by means of the magneiztable particles associated with the balloon 301 or the device 200.

The retrieval device 930 is inserted into the body cavity. After opening the flanges 831 of the retrieval device, the engaging probe 932 with magnetizable portion 929 in its tip is inserted into the body cavity so as to engage a magnet associated with either the device 200 or the balloon 301. The probe 932 is then pulled so as to bring the balloon 301 into the grip of flanges 831 of the retrieval device 930. A piercer 933 is inserted into the balloon 301 to drain the fluid 724 contained in its lumen 304 into an attached syringe (not shown) or into the body cavity. The retrieval device 930 is then withdrawn from the individual together with the device 200 and the deflated balloon 301.

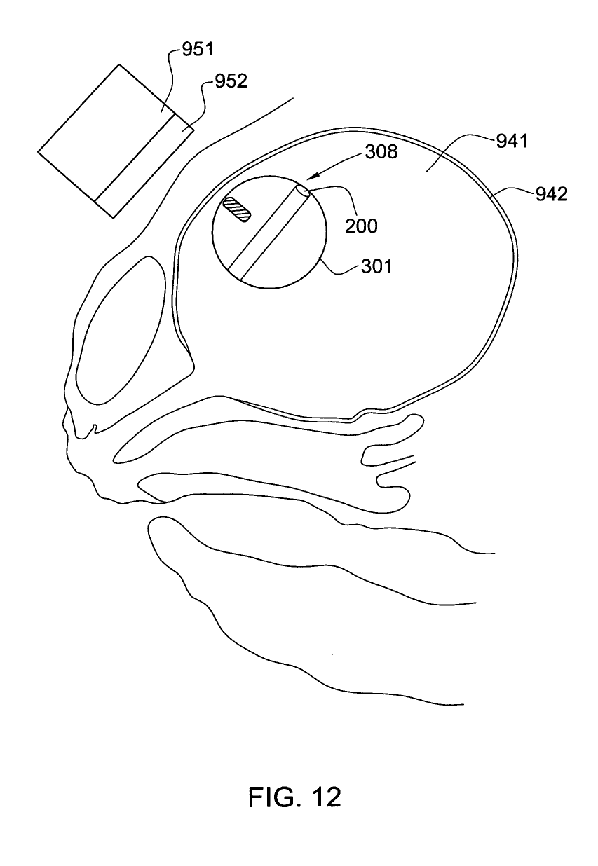

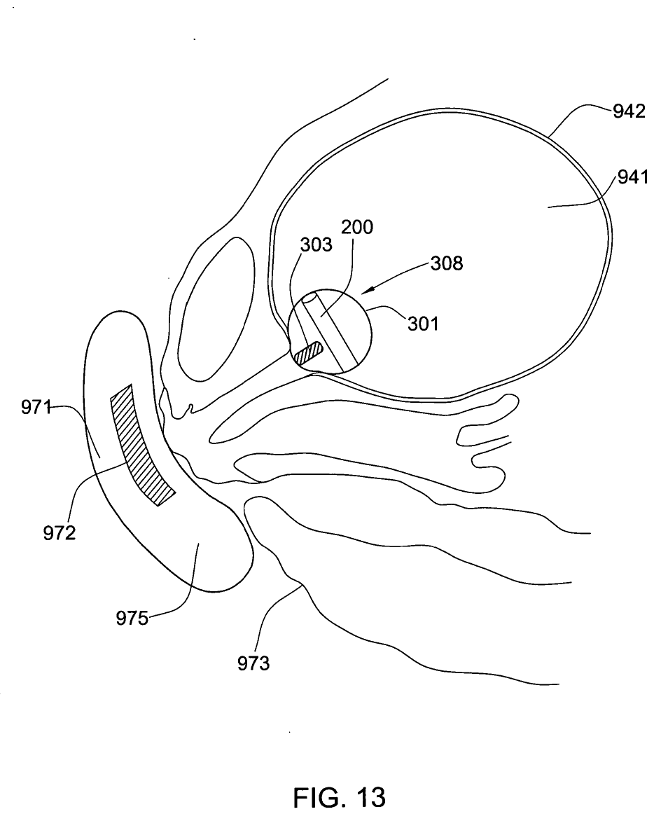

FIG. 12 shows use of a displacing member 951 to position the device-balloon combination 308 at a desired location in the body cavity. The displacing member 951 is located outside the individual's body and comprises a magnetizable portion 952. The displacing member 951 is placed at a location on the surface of the individual's body so as to draw the device balloon combination to a desired location within the body cavity.

FIG. 13 shows use of an immobilizing member 971 comprising a magnetizable portion 972 affixed to the surface 973 of the individual's body so as to maintain the device-balloon combination 308 at the desired location in the body cavity. The magnetizable portion 972 of immobilizing member 971 may be enclosed in a coating 975 so as to form, for example, a hygienic pad. The immobilizing member 971 may be affixed to the surface 73 by means of tape, or may be incorporated into a garment worn by the individual.

The invention has been described with a certain degree of particularly only for the sake of clarity. However, several variations and modifications in the invention are possible without exceeding the scope and spirit of the invention as defined in the following set of claims.

1. A medical device for controlled release of one or more substances into a body cavity containing an electrolytic fluid comprising:
(a) a power supply having first and second terminals;
(b) a plurality of blister-like vesicles mounted on a first surface, each vesicle having at least a metallic portion formed from a first metal;
(c) for each vesicle, an electrical connection between the metallic portion of the vesicle and the first terminal of the power supply, each connection including a switch so as to allow the metallic portion to function as an anode when the switch is closed; and
(d) A cathode formed from a second metal attached to the second terminal of the power supply;
wherein the cathode is separated from the anodes by a space that is assessable by the electrolytic fluid when the device is in the body cavity.

2. The device according to claim 1 further comprising a processor configured to close one or more switches at one or more predetermined times.

3. The device according to claim 1 further comprising one or more magnetizable particles.

4. The device according to claim 1 wherein the switches are closed by means of a remote control.

5. The device according to claim 1, wherein the body cavity is a urinary bladder or a digestive tract organ.

6. The device according to claim 1 wherein the anodes are formed from copper and the cathode is formed from zinc.

7. The device according to claim 1 further comprising an inflatable balloon.

8. The device according to claim 7, wherein the balloon is formed with a magnetizable portion.

9. The device according to claim 7 in which the balloon further comprises a self-sealing valve.

10. The device according to claim 7, wherein the device after inflation of the balloon floats in the electrolytic fluid.

11. The device according to claim 7, wherein the device after inflation of the balloon sinks in the electrolytic fluid.

12. The device according to claim 1 wherein one or more of the one or more substances are drugs or antibiotics.

13. The device according to claim 1 wherein one or more of the one or more substances are radioactive substances.

14. The device according to claim 1, further comprising one or more monitoring devices for parameters in the body cavity.

15. The device according to claim 14, wherein one or more of the one or more of the monitoring devices monitors a parameter of the body cavity selected from the list comprising:
(a) pressure of the electrolytic fluid;
(b) temperature of the electrolytic fluid;
(c) density of the electrolytic fluid; and
(d) composition of the electrolytic fluid.

16. The device according to claim 14 further comprising a processor configured to receive data from a monitoring device and to close one or more switches when under predetermined conditions in the body cavity.

17. A system for treating a body cavity of an individual, the system comprising:
(a) a device according to claim 1; and
(b) an applicator for inserting the device into the body or for removing the device from the body cavity, the applicator fitted at an end thereof with a gripping device for releasably gripping the device;

18. A system for treating a body cavity of an individual, the system comprising:
(a) a device according to claim 7;
(b) an applicator for inserting the device into the body or for removing the device from the body cavity, the applicator fitted at an end thereof with a gripping device for releasably gripping the device; and
(c) an inflating device for inflating the balloon.

19. The system according to claim 17 further comprising a magnetizable displacing member for displacing the device within the body cavity.

20. The system according to claim 17, further comprising an immobilizing member comprising a magnetizable portion, said immobilizing member being secured onto the individual's body for immobilizing the device at a desired location in the body cavity.

21. The system according to claim 20, wherein the immobilizing member is in the form of a hygienic pad configured to be placed in a garment of the individual.

22. The system according to claim 17, wherein the gripping device comprises flanges.

23. The system according to claim 17, wherein the gripping device comprises a magnetizable portion.

24. The system according to claim 18, wherein the inflating device comprises an injector for injecting a fluid into the balloon so as to expand the balloon.

25. A method for releasing one or more substances into a body cavity containing an electrolytic fluid of an individual comprising the steps of:
(a) loading the one or more substances into the vesicles of a device according to claim 1;
(b) inserting the device into the body cavity;
(c) expanding the balloon in the urinary bladder; and
(d) displacing the balloon within the urinary bladder to a desired location.

26. A method for releasing one or more substances into a body cavity containing an electrolytic fluid of an individual comprising the steps of:
(a) loading the one or more substances into the vesicles of a device according to claim 7;
(b) inserting the device into the body cavity; and
(c) expanding the balloon in the body cavity.

27. The method according to claim 25 further comprising displacing the device within the body cavity to a desired location.

28. The method according to claim 25 wherein one or more of the one or more substances are selected from the list comprising:
(a) drugs;
(b) immunoglobulins
(b) antibiotics; and
(c) radioactive substances.
